(19) United States
(12) Patent Application Publication
(10) Pub. No.: US 2007/0232872 A1
(43) Pub. Date: Oct. 4, 2007

(54) CONTINUOUS NONINVASIVE GLUCOSE MONITORING IN DIABETIC, NON-DIABETIC, AND CRITICALLY ILL PATIENTS WITH OCT

(75) Inventors: Donald Prough, Galveston, TX (US); Rinat O. Esenaliev, League City, TX (US); Massoud Motamedi, Houston, TX (US)

Correspondence Address: ROBERT W STROZIER, P.L.L.C, PO BOX 429, BELLAIRE, TX, 77402-0429, US

(73) Assignee: The Board of Regents of The University of Texas System, Austin, TX (US)

(21) Appl. No.: 11/685,574

(22) Filed: Mar. 13, 2007

Related U.S. Application Data
(60) Provisional application No. 60/782,904, filed on Mar. 16, 2006.

(51) Int. Cl. A61B 5/00

(52) U.S. Cl. 600/316

(57) ABSTRACT

New optical coherence tomography (OCT) techniques are disclosed which are designed to improve OCT glucose concentration measure accuracy and are capable of being performed on a continuous basis. New multi-wavelength optical coherence tomography (OCT) techniques are also disclosed and designed to reduce artifacts do to water. New optical coherence tomography (OCT) techniques are also disclosed for determining local profusion rates, local analyte transport rates and tissue analyte transport rates as a measure of tissue health, disease progression and state and tissue transplantation effectiveness.

### RELATED APPLICATIONS

This application claims priority to United State Provisional Application Ser. 60/782,904; FD: Mar. 16, 2006.

### BACKGROUND OF THE INVENTION

1. Field of the Invention

The present invention relates to a method for continuous noninvasive glucose monitoring in an animal including an human using an optical coherence tomography (OCT) based glucose monitoring system.

More particularly, the present invention relates to a method for continuous noninvasive glucose monitoring in an animal including an human using an OCT based glucose monitoring system. The method includes the step of generating radiation. A first portion of radiation is directed to a plurality of locations (a plurality of 1-D scans) of a tissue site to generate back-scattered and/or reflected radiation, where a distance between any two locations is between 500 nm and 20 mm. A second portion of the radiation is directed to a reflector to generate reference radiation. The back-scattered and/or reflected radiation and the reference radiation are then detected to produce optical coherence tomography signals. A glucose concentration is then calculated using a composite slope of the optical coherence tomography signals, where the number of signals (1-D scans) is sufficient to improve the signal-to-noise ratio of a composite OCT signal improving the OCT derived glucose concentration.

2. Description of the Related Art

In both diabetic and nondiabetic patients, hyperglycemia and insulin resistance commonly complicate critical illness [1-5]. In critically ill patients, even moderate hyperglycemia contributes to complications [4-8]. In diabetic patients with acute myocardial infarction, maintenance of blood glucose concentration ([Glu_{b}])<215 mg/dL (11.9 mmol/L) improved mortality at one year and 3.5 years [9-11].

In a recent clinical trial of human growth hormone to reduce catabolism in critically ill patients, mortality was doubled in the treatment group [12], perhaps because of growth-hormone induced hyperglycemia [13]. In 1548 patients (87% of whom were non-diabetic) randomized to receive conventional management or intensive insulin therapy to tightly control [Glu_{b}] between 80 and 110 mg/dL, intensive insulin therapy reduced mortality by more than 40% (from 8.0% to 4.6%) but carried a 5.0% risk of inducing severe hypoglycemia ([Glu_{b}]<40 mg/dL) [13]. Therefore, in critically ill patients, continuous glucose monitoring, ideally noninvasive, would be invaluable to guide insulin infusion to both control hyperglycemia and avoid hypoglycemia. However, no suitable noninvasive device is available.

U.S. Pat. No. 6,725,073 issued Apr. 20, 2004 disclosed a methods for measuring analyte concentration within a tissue using optical coherence tomography (OCT), incorporated therein by reference here and as set forth comprehensively below. Radiation is generated, and a first portion of the radiation is directed to the tissue to generate backscattered radiation. A second portion of the radiation is directed to a reflector to generate reference radiation. The backscattered radiation and the reference radiation is detected to produce an interference signal. The analyte concentration is calculated using the interference signal. This patent of two of the inventors set forth the basic principals of OCT and the reader is directed thereto for additional details of the OCT system. However, the method of U.S. Pat. No. 6,725,073 has not readily amenable to continuous monitoring.

Thus there is a need in the art for a noninvasive reliable method and system of continuously monitoring glucose concentration in patients in order to control glucose concentration so as not to induce hyperglycemia or hypoglycemia, especially in critically ill patients.

### SUMMARY OF THE INVENTION

The present invention also provides a method for continuous noninvasive glucose monitoring in an animal including an human using an OCT based glucose monitoring system. The method includes the step of generating radiation. A first portion of radiation is directed to a plurality of locations (a plurality of 1-D scans) of a tissue site to generate back-scattered and/or reflected radiation. A second portion of the radiation is directed to a reflector to generate reference radiation. The back-scattered and/or reflected radiation and the reference radiation are then detected to produce optical coherence tomography signals. A glucose concentration is then calculated on a continuous basis or periodic basis using a composite slope of the optical coherence tomography signals, where the number of signals is sufficient to improve the signal-to-noise ratio of a composite OCT signal improving the OCT derived glucose concentration. In certain embodiments, the method is directed to 1-D scans of a tissue site that does not have inhomogeneities over the area in which the 1-D scans are taken. In certain embodiments, the plurality of 1-D scans are directed over a tissue are having an area between about 200μ×200μ and about 2000μ×2000μ. In other embodiments, a distance between any pair of 1-D scans is between about 500 nm and 20 mm. In other embodiments, the distance between any pair of 1-D scans is between 1 μm and 10 mm. In certain embodiments, the area is chosen such that tissue structures having OCT characteristics that permit reliable and reproducible glucose concentration measurements. Some of the tissue characteristics that give rise to such “stable” OCT glucose measurements are continuous and/or contiguous layers, morphological properties, a degree of vascularization of the tissue or layers therein, analyte transport properties, etc.

The present invention also provides a method for continuous noninvasive glucose monitoring in an animal including an human using an OCT based glucose monitoring system. The method includes the step of generating radiation. A first portion of radiation is directed onto a surface of a tissue site to generate back-scattered and/or reflected radiation. A second portion of the radiation is directed to a reflector to generate reference radiation. The back-scattered and/or reflected radiation and the reference radiation are then detected to produce optical coherence tomography signals. A glucose concentration is then calculated on a continuous basis or periodic basis using a composite slope of the optical coherence tomography signals over the surface, where the number of signals is sufficient to improve the signal-to-noise ratio of a composite OCT signal improving the OCT derived glucose concentration.

The present invention also provides a method for continuous noninvasive glucose monitoring in critically ill patients. The method includes the step of generating radiation. A first portion of radiation is directed to a plurality of locations of a mucosa such as an oral mucosa of the patient to generate back-scattered and/or reflected radiation. A second portion of the radiation is directed to a reflector to generate reference radiation. The back-scattered and/or reflected radiation and the reference radiation are then detected to produce optical coherence tomography signals. A glucose concentration is then calculated on a continuous basis or periodic basis using a composite slope of the optical coherence tomography signals, where the number of signals is sufficient to improve the signal-to-noise ratio of a composite OCT signal improving the OCT derived glucose concentration. The method can also include the step of using glucose concentration values obtained from invasive samplings of blood (routinely used in critically ill patients) to calibrate the OCT-based sensor and improve OCT glucose concentration accuracy. The method is especially well suited for patients undergoing cardiac surgery, where careful control of glucose level leads to a substantial reduction in mortality and morbidity of in such patients. The inventors believe that probing of mucosa may provide more accurate glucose monitoring due to better blood perfusion and glucose transport compared in the mucosa as compared to skin mucosa

The present invention provides an OCT system including a light source, a optical subsystem adapted to produce a reference beam and a sample beam. The optical subsystem is also configured to direct the sample beam onto a plurality of sites of a tissue or to direct the sample beam over an area of a tissue producing a plurality of 1-D OCT scans on a continuous basis or periodic basis. The optical subsystem also includes an interferometer for combining the reference beam and a back-scattered beams from each sample scan and directing the combined beams to a photodetector adapted to collect plurality of combined beams and produce a plurality of OCT signals which are then transferred to an analyzer as they are collected. The analyzer is designed to accumulate the plurality of 1-D scans and produce a composite OCT signal with improved signal-to-noise ratio and to produce a slope of the OCT composite signal and to derive a corresponding OCT glucose concentration. The analyzer can also be designed to receive invasive blood glucose data taken during the continuous monitoring time to improve OCT software calibration and signal registration.

The present invention provides a computer readable media containing program instructions for measuring glucose concentration of a plurality of 1-D scan of a tissue area. The computer readable media including instructions for storing a plurality of 1-D optical coherence tomography (OCT) signals in memory. The computer readable media also includes instructions for combining the signals into a composite signal with an improved signal-to-noise ratio. The computer readable media also includes instructions for determining the glucose concentration within the tissue using the composite signal. The instructions for determining the glucose concentration include determining a slope of the composite OCT signal and determining a OCT glucose concentration within the tissue using the slope. The computer readable media can also include instructions to identify structures within the tissue area at a given depth in the tissue which improve the OCT glucose concentration value relative to the actual blood glucose concentration in the tissue.

The present invention provides a computer readable media containing program instructions for continuously measuring glucose concentration of a plurality of 1-D scan of a tissue area. The computer readable media includes instructions for storing a plurality of 1-D optical coherence tomography (OCT) signals in memory, instruction of forming a composite OCT signal from the plurality of 1-D scans and instructions for determining the glucose concentration within the tissue using the composite signal. The instructions for determining the glucose concentration include instructions for correlating a change in the slope with an optical or morphological change in the tissue. The computer readable media can also include instructions to identify structures within the tissue area at a given depth in the tissue which improve the OCT glucose concentration value relative to the actual blood glucose concentration in the tissue.

Besides deriving reliable, continuous glucose concentration values from the slope of the backscattering signal across the entire depth of tissue scanned in a 1-D scan, reliable and continuous glucose concentration also is derivable from other information contained in the backscatter signal. Reliable glucose concentrations can be derived from portion of the signal or from a collection of binned signal data. In scan including a plurality of 1-D scans, the glucose concentration can be derived from randomly or pattern selected 1-D scan or portions thereof, randomly or pattern selected 1-D scans or portions thereof, or any other combination of signal data derived from the plurality of ID scans.

The present invention also provides methods for scanning a tissue site including the step of directly an OCT sample beam onto a plurality of locations of an area of a tissue so that each OCT signal is an in-depth scan of the location, a so-call A-scan. The plurality of locations can include a random collections of individual locations within the area. The plurality of locations can include a patterned selection of individual locations within the area. The plurality of locations can include a random selection of contiguous subareas. The plurality of locations can include a patterned selection of contiguous subareas. The plurality of locations can include the entire area. Thus, an A-scan method collects in-depth 1-D scans at a plurality of locations within the tissue area, where the mirror in the reference beam path is moved to change the sample beam penetration depth, i.e., an entire depth profile is scanned at each location.

The present invention also provides methods for scanning a tissue site including the step of directly an OCT sample beam onto a plurality of locations of an area of a tissue so that each OCT signal is scanned at a given depth at each location, a so-call C-scan. The plurality of locations can include a random collections of individual locations within the area. The plurality of locations can include a patterned selection of individual locations within the area. The plurality of locations can include a random selection of contiguous subareas. The plurality of locations can include a patterned selection of contiguous subareas. The plurality of locations can include the entire area. Thus, a C-scan method collects single depth 1-D scans at a plurality of locations within the tissue area, where the mirror in the reference beam path is fixed at a given penetration depth.

The present invention also provides methods for scanning a tissue site including the step of directly an OCT sample beam onto a plurality of locations of an area of a tissue so that each OCT signal is simultaneously depth and laterally varied. The plurality of locations can include a random collections of individual locations within the area. The plurality of locations can include a patterned selection of individual locations within the area. The plurality of locations can include a random selection of contiguous subareas. The plurality of locations can include a patterned selection of contiguous subareas. The plurality of locations can include the entire area. Thus, the new scan method collects scans at a plurality of locations within the tissue area at varying depth and locations by simultaneously moving the beam over the surface to adjust the location and moving the mirror to adjust the signal depth being scanned.

Regardless of the method of scanning, the methods will ultimately convert to a single OCT composite glucose concentration value. Again the size of the plurality of locations is sufficient to produce a composite signal (averaged, binned-averaged, etc.) that has improved signal-to-noise ratio and/or improved sensitivity.

The area to be scanned can be a regular area or an irregular area. The regular area are generally geometrical areas such as polygonal areas such as triangular areas, quadrilateral areas, pentagonal areas, hexagonal areas, etc.

The present invention also provides a method for characterizing tissue based on measurements of water transport and/or other analyte transports into and out of a tissue site. The method can be used to determine a state of a disease measuring analyte flow into and out the tissue site. For example, tissue of a patient in an early stage of a chronic illness such as diabetes has different analyte transport compared to tissue of a patient in a late stage of a chronic illness. Such characterization can be used to improve diagnosis, improve disease progress diagnosis, improve determination of treatment efficacy, and improve an understanding of tissue transport properties at different stages of a disease. For example, in cornea transplants, water transport through the tissue being used in the transplant and post transplantation are properties that correlate with transplant efficacy.

The present invention also provide a method for measuring blood profusion in the tissue using a Doppler OCT probe. The Doppler data can give information about blood profusion, water transport, and analyte transport. The Doppler data can used to depth profile the tissue to determine optimal location in the tissue for collecting OCT glucose data, where tissue regions with higher blood profusion rates will generally give rise to improved OCT glucose concentration measurement. Thus, the present invention also provide a method for identifying tissues structures that are more suited OCT glucose monitoring.

The present invention also provides multiwave length OCT, where one or more wavelengths (single wavelength or narrowly banded wavelength narrow wavelength bandwidth) are used in OCT scanning. The scanning method can include performing a first 1-D scan at a location at a first frequency and then a second 1-D scan at the same location at a second frequency. The method can include making additional 1-D scans at other frequencies as well, but generally the inventors believe that two wavelength are sufficient if judiciously selected. Alternatively, the method can include scanning a portion or all of a tissue area at a first wavelength and then scanning the same or different portion or all of the tissue area with a second wavelength. The wavelength are selected from the electromagnetic spectrum between about 700 and about 2000 nm. In certain embodiments, the first wavelength is a longer wavelength generally between about 1300 nm and about 2000 nm and the second wavelength is a shorter wavelength generally between about 700 nm and 1300 nm. The longer wavelength data correlates with water contributions to the OCT signal and the longer wavelength data is thus used to correct the OCT data at shorter wavelength, which generally correlates between glucose contributions to the OCT signal. The longer wavelength OCT signals are more water specific allowing efficient removal of water contributions, while shorter wavelength improve contrast. The combination of the two signal types can be used to enhance glucose specificity by better accounting for artifacts do to water. Alternatively, the OCT scan can be collected at one or more glucose specific wavelengths, but currently no light source are commercially available that generate light at those wavelengths. The two wavelength specific signals can be combined using an acceptable mathematical technique such as ratiometric analysis.

The present invention also provides OCT system that are equip with Doppler capability so that profusion rates, water transport rates and/or other analyte transport rates. The OCT system of this invention can also include adaptive optics. Adaptive optics are optics that are designed to minimize the aberration of light passing through the tissue or other light propagation artifacts by reactively adjust aspects of the sample beam such as beam contour, beam focus, beam angle of incident, etc., which provide optimal OCT signal with improved sensitivity or signal-to-noise ratio.

### BRIEF DESCRIPTION OF THE DRAWINGS

The invention can be better understood with reference to the following detailed description together with the appended illustrative drawings in which like elements are numbered the same.

FIG. 1A depicts a schematic diagram of an embodiment of an Optical Coherence Tomography (OCT) system of this invention.

FIG. 1B depicts a schematic diagram of a fiber optics embodiment of an Optical Coherence Tomography (OCT) system of this invention, with optional adaptive optics system.

FIG. 2 depicts a plot of the slope of OCT signals recorded from a human subject and blood glucose concentration measured at different time during oral glucose tolerance test (OGTT). Blood glucose concentration was measured every 15 minutes.

FIG. 3 depicts a plot of the slope of OCT signals recorded from a human subject and blood glucose concentration measured at different time during OGTT. Blood glucose concentration was measured every 5 minutes.

FIG. 4A depicts a plot of the slope of OCT signals obtained from rabbit ear during glucose clamping experiment with scanning over 0.2 mm×0.2 mm area.

FIG. 4B depicts a plot of the slope of OCT signals obtained from the rabbit ear with scanning over 0.2×0.2 mm area vs. blood glucose concentration.

FIG. 5A depicts an OCT image of pig skin; 1 and 2 show the layers in which the correlation coefficients between the OCT signal slope and blood glucose concentration were highest.

FIG. 5B depicts a corresponding histological section of pig skin; 1 and 2 demonstrate the layers at which the correlation coefficients between the OCT signal slope and blood glucose concentration were highest.

FIG. 6 depicts an OCT signal slope, blood glucose concentration and blood sodium (Nan) concentration vs. time.

FIG. 7A depicts an OCT signal slope, blood potassium (K⁺) concentration, blood chloride (Cl⁻) concentration and blood urea concentration vs. time.

FIG. 7B depicts an OCT signal slope, pH, pCO₂, and hematocrit (Hct) vs. time.

FIG. 8 depicts the transport of water within cornea following the application of one drop of water on the surface of rabbit cornea in vivo.

FIG. 9 depicts the transport of water within rabbit cornea in vivo following the application of one drop of dextrose which is known to cause cornea dehydration and shrinkage.

### DETAILED DESCRIPTION OF THE INVENTION

The inventors have developed a novel optical coherence tomography (OCT) technique for noninvasive, continuous glucose monitoring based on interferometric measurement and analysis of low-coherent light back-scattered from specific layers of a tissue for use in diabetic and non-diabetic patients, especially critically ill patients or critical care patients. The inventors have identified specific tissue layers in which a correlation between a OCT signal slope and a blood glucose concentration is highest. The inventors have also found that blood glucose concentrations can be monitored using OCT using low-coherence interferometry (LCI) by probing specific layers within a tissue area. The term substantially similar morphology means that the morphology of the tissue over the area that is to be scanned by the OCT probe does not change substantially or alternatively, the tissue layer as continuous, morphologically similar and contiguous. The inventors have found that the tissue area most suited for accumulating a plurality of 1-D OCT scans within the area is generally between about 200μ×200μ and about 2000μ×2000μ. Alternatively, the locations of the 1-D OCT scans are selected so that the locations are between 500 nm and 20 mm. The inventors have found that a plurality of 1-D OCT scans of such an area in a random pattern or a progressive pattern can be used to construct a 3-D glucose concentration profile for the tissue two dimension representing an area of tissue and the third dimension representing the penetration depth of the back-scattered sample beam.

The present invention relates broadly to a method for continuously monitoring glucose concentration in an animal including an human. The method includes the step of generating radiation. A first portion of the radiation is directed to a plurality locations of an area of a tissue site to generate back-scattered and/or reflected radiation, where the area comprises a tissue site, where the tissue is substantially similar across the area. A second portion of the radiation is directed to a reflector to generate reference radiation. The back-scattered and/or reflected radiation and the reference radiation are then detected to produce optical coherence tomography signals. A glucose concentration is then calculated using a slope of the optical coherence tomography signals, where the number of signals is sufficient to improve the signal-to-noise ratio of a composite OCT signal improving the OCT derived glucose concentration. The method is ideally suited for use with mucosa tissue site, where the inventors believe that more accurate glucose monitoring is possible due to better blood perfusion and glucose transport in mucosa compared to skin. The method can also include the step of using glucose concentration values obtained from initial invasive contemporary samplings of blood (routinely used in critically ill patients) in order to calibrate the OCT-based sensor to improve OCT glucose accuracy.

The method has been successfully used to continuously and noninvasively monitor glucose concentration in animals and humans by probing skin, oral mucosa, and other tissues, using an OCT signal slope measurements for glucose monitoring. The method is also ideally suited for glucose monitoring systems in the field such as in military settings, mass-causality settings, other high causality settings or in emergency care vehicles. To minimize motion artifacts and improve accuracy, the OCT system beam is adapted to scan over a 2-D lateral area of the tissue, where the area is a square, rectangular, circle, elliptical, or other 2-D area. The inventors have found that the OCT signal obtained with 2-D scanning is more stable compared with that obtained with standard linear (1-D) lateral scanning as disclose in U.S. Pat. No. 6,725,073. The inventors have demonstrated that glucose monitoring can be performed with high accuracy and specificity if an optimal lateral scanning mode is used and specific layers of skin are probed. The inventors have also demonstrated that variation of concentrations of major blood osmolytes [Na⁺], [K⁺], [HCO₃⁻], urea, pH, and PCO₂ did not influence significantly the OCT signal slope. The inventors have also demonstrated that a best correlation of OCT signal slope can be obtained in specific tissue layers, in particular, near a dermis-subdermis boundary and near a papillary and reticular junction in the dermis. The inventors have found that the system can be used to monitor blood glucose concentration by using low-coherence interferometry with probing these specific layers.

The system of the present invention also includes components adapted to improve the sensitivity and accuracy of OCT non-invasive glucose monitoring in tissue. The components include integrating adaptive optics and engineering the optics of the OCT probe to confine the volume of tissue probed by OCT. By confining the irradiated volume, a quality of imaging can be improved within a specific region of tissue. The ability to confine the irradiation volume permits specific regional probing of glucose-induced changes in OCT signal by monitoring a glucose concentration in a pre-determined volume of tissue within a specific layer of skin or mucal structures. The components can also include an OCT-Doppler component to acquire quantitative measurements of changes in local perfusion rates within the targeted layer in order to perform simultaneous monitoring of changes in a local perfusion rate, a local glucose concentration and a local water concentration within the predetermined volume of tissue. The Doppler component can also be use to guide selection of a region where optical probing of tissue could be performed within a layer of tissue that may be closer or further away from vascular network of tissue that can be localized using high resolution OCT-Doppler. The components can also include a multi-wavelength light and OCT measurements at different wavelengths to allow correction for changes in OCT slope induced by changes in water content in the target tissue as fluid loading is known to play a significant role in the management of critical care patient. Furthermore, selection of a spectral region for an OCT light source that can allow quantitative monitoring of tissue water content and glucose concentration would be useful in the management of critically ill patients. Multi-spectral OCT imaging or spectroscopy can also be deployed to correct for artifacts that may be induced by changes in hematocrit concentration during an OCT measurement. A combination of OCT sensing and functional measurements may provide a unique approach for monitoring of critically ill patients.

Glucose monitoring is very important in critically ill non-diabetic and diabetic patients. OCT technique is capable of continuous glucose monitoring making it well suited for use in intensive care units (ICUs), emergency departments, field, military and mass casualty settings, and emergency care vehicles. The invention can also be used by non-medical personnel too (with minimal training) especially in the filed, military and mass-casualty settings, because the OCT systems are noninvasive.

OCT signal slope is not being used or proposed for continuous glucose monitoring in critically ill patients. This invention is not obvious to a person having ordinary skill in the art to which this invention pertains. It is necessary to understand and demonstrate why OCT signal slope is dependent on glucose concentration and water content of tissue depending on what region of the spectra is used for optical probing of tissue. The inventors have demonstrated that this technology is capable of glucose monitoring in phantoms and in vivo in animals and humans. The inventors have also demonstrated that OCT can quantitatively monitor the changes in tissue hydration and can provide a non-invasive mean for monitoring of water transport in corneal tissue suggesting that the influence of water on OCT measurements can be accounted by performing the proposed measurements in spectral region with dominant dependence on optical properties of water as compared to region where attenuation of probing light by tissue water content is less significant.

This is a novel approach for quantitative continuous noninvasive monitoring of changes in glucose concentration, water content and local perfusion in a predetermined region of tissue that significantly improve the ability to manage care for critically ill patients. The inventors have found no publications or patents on noninvasive continuous glucose and hydration monitoring with OCT in critically ill patients.

One embodiment of this invention is the use of continuous noninvasive glucose monitoring in critically ill patients to tightly control glucose concentrations in the patients between 80 and 110 mg/dL significantly reducing mortality.

In both nondiabetic and diabetic patients, hyperglycemia and insulin resistance commonly complicate critical illness [1-5]. Even moderate hyperglycemia, at levels that conventionally have not been treated acutely with insulin because of the risk of inducing hypoglycemia, contributes to morbidity and mortality [4-8]. Stress-induced hyperglycemia is associated with poorer outcome in both nondiabetic and diabetic patients after stroke [15] and acute myocardial infarction [16]. Until recently, hyperglycemia was recognized as a common laboratory abnormality in critically ill patients but was not regarded as an important factor contributing to (rather than associated with) poor outcome. In general, hyperglycemia has been considered to be a secondary response to stress and infection and not an independent risk factor for poor outcome; recently, however, evidence has increased that hyperglycemia is in fact a risk factor for poor outcome [17, 18]. In nondiabetic patients with protracted critical illnesses, high serum levels of insulin-like growth factor-binding protein 1, which reflect an impaired response of hepatocytes to insulin, increase the risk of death [19, 20].

Despite considerable interest in the influence of tight glycemic control in outpatients on the incidence and severity of the chronic microvascular and macrovascular complications of diabetes [21-26], most clinicians until recently have loosely controlled blood glucose in critically ill patients. The reasoning behind loose control in critically ill patients included several considerations: 1) assumed low likelihood that a short interval of poor glycemic control would significantly influence the progression of chronic complications; 2) difficulty of anticipating insulin requirements in patients with unstable levels of stress; and 3) risk of inducing hypoglycemia with excessive insulin therapy. Subsequently, several investigators have performed clinical trials of tight glucose control in both diabetic and nondiabetic patients with a variety of critical illnesses. In diabetic patients with acute myocardial infarction, maintenance of blood glucose concentration ([Glu_{b}])<215 mg/dL (11.9 mmol/L) improved mortality at one year and 3.5 years [9-11]. In 1548 patients (87% of whom were nondiabetic) randomized to receive conventional management or intensive insulin therapy to tightly control [Glu_{b}] between 80 and 110 mg/dL, intensive insulin therapy reduced mortality from 8.0% to 4.6% but carried a 5.0% risk of inducing severe hypoglycemia ([Glu_{b}]<40 mg/dL) [13]. In a recent clinical trial of human growth hormone to reduce catabolism in critically ill patients, mortality was doubled in the treatment group [12], perhaps because of growth-hormone induced hyperglycemia [13]. Therefore, in critically ill patients, continuous glucose monitoring, ideally noninvasive, would be invaluable to guide insulin infusion to both control hyperglycemia and avoid hypoglycemia. However, no suitable noninvasive device is available.

The impact of tight glucose control during critical illness in nondiabetic patients is best appreciated from a more detailed description of the landmark paper by Van de Berghe et al. [13]. The authors hypothesized that hyperglycemia or relative insulin deficiency (or both) during critical illness directly or indirectly predisposed patients to complications [6, 27, 28], such as severe infections, polyneuropathy, multiple-organ failure, and death. In addition to the reduction that they achieved in total mortality from 8.0% with conventional treatment to 4.6% with intensive insulin therapy, other secondary analyses also are important. The greatest reduction in mortality involved deaths due to multiple-organ failure with a proven septic focus. Intensive insulin therapy also reduced overall in-hospital mortality by 34%, bloodstream infections by 46%, acute renal failure requiring dialysis or hemofiltration by 41%, the median number of red-cell transfusions by 50%, and critical-illness polyneuropathy by 44%, and patients receiving intensive therapy were less likely to require prolonged mechanical ventilation and intensive care. Much of the benefit of intensive insulin therapy was attributable to its effect on mortality among patients who remained in the ICU for more than five days (20.2% vs. 10.6% with intensive insulin therapy).

However, tight glycemic control also was associated with complications. Intensive insulin therapy carried a 5.0% risk of inducing severe hypoglycemia (blood glucose <40 mg/dL) [13]. The authors state that these episodes of hypoglycemia were not associated with other morbidity, but it is unlikely that experience outside the rigorously controlled environment of a single-institution clinical trial would be so favorable. Ideally, insulin therapy should be adjusted during continuous infusion so that blood glucose is not permitted to decrease below 60 mg/dL; that degree of precision is difficult to avoid with intermittent glucose sampling and requires very frequent, ideally continuous monitoring.

In the discussion, Van de Berghe et al. [13] noted that glycemic control compares highly favorably to other interventions that have been proposed to improve survival of critically ill patients. One of the few to improve survival is treatment of sepsis with the highly expensive intervention, activated protein C, which only reduced mortality by 20% at 28 days [29]. In contrast, tight glycemic control is applicable to a much larger proportion of critically ill patients and reduced mortality by more than 40% [13]. Intensive insulin therapy also reduced the utilization of expensive intensive care resources and the risk of complications, including episodes of septicemia and the associated need for prolonged antibiotic therapy. Tight glycemic control may reduce the deleterious effects of hyperglycemia on macrophage or neutrophil function [30-33] or support insulin-induced trophic effects on mucosal and skin barriers. Intensive insulin treatment also reduced the incidence of acute renal failure, a particularly morbid and highly lethal complication of critical illness. The reduced number of transfusions in the intensive-treatment group may reflect improved erythropoiesis or reduced hemolysis, since this benefit was associated with a lower incidence of hyperbilirubinemia. Alternatively, intensive insulin therapy may reduce the risk of cholestasis, since adequate provision of glucose and insulin to hepatocytes is crucial for normal choleresis [34, 35].

The inventors conclude that evidence strongly indicates that intensive insulin therapy to maintain blood glucose between 80 and 110 mg/dL reduces morbidity and mortality among critically ill patients in the surgical intensive care unit [13], but that evidence also indicates an important risk of inducing hypoglycemia. Thus, in critically ill patients, continuous glucose monitoring, ideally noninvasive, is invaluable to guide insulin infusion to both control hyperglycemia and avoid hypoglycemia.

Thus, the present invention relates to the use of the OCT technique for noninvasive glucose monitoring in these groups of patients which include diabetic and nondiabetic critically ill patients with a variety of conditions: trauma patients, surgical (including cardiac surgery) patients, patients with sepsis, etc.

Several scientific groups have been developing noninvasive techniques for blood glucose monitoring using various optical approaches including polarimetry [36, 37], Raman spectroscopy [38, 39], near infrared (NIR) absorption and scattering spectroscopy [40-44], and optoacoustics [45-46]. Despite significant efforts, these techniques have limitations associated with low sensitivity, accuracy, and insufficient specificity of glucose concentration measurement within the relevant physiological range (4-30 mM or 72-240 mg/dL). Invasive devices for home use have a reported accuracy of 20%, i.e., a true [Glu_{b}] of 5 mM would measure between 4 and 6 mM. A noninvasive glucose sensor should have a comparable accuracy.

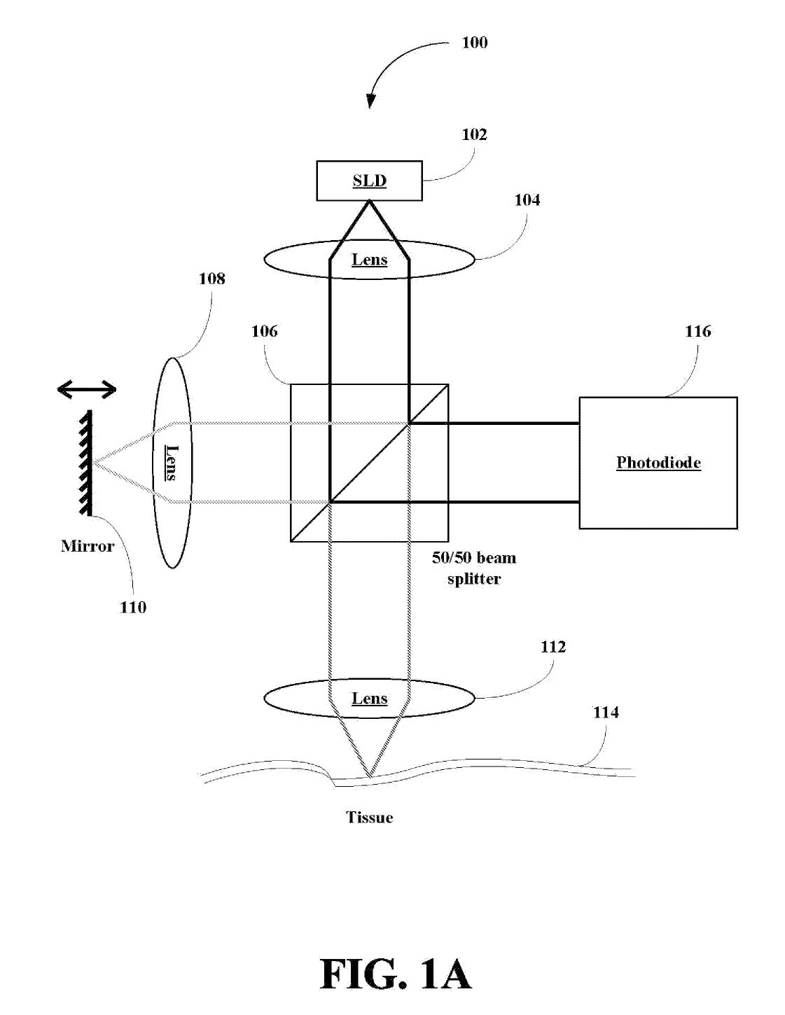

OCT is a new optical diagnostic technique that provides depth resolved images of tissues with resolution of about 10 μm or less at depths of up to 1 mm. The present invention is directed to the use of the OCT technique for monitoring of blood glucose concentration by measuring and analyzing light coherently backscattered from specific tissue layers as demonstrated in animal and clinical studies to continuously, noninvasively and accurately monitoring glucose monitoring [46-53]. The basic principle of the OCT technique is to detect back-scattered photons from a tissue of interest within a coherence length of a light source using a two-beam interferometer. A OCT system for use in this invention, generally 100, is shown in FIG. 1A. Light from a superluminescent diode (SLD), a light source with low coherence, 102 passed through a first lens 104 and then directed to a 50/50 beam splitter 106. Half of the beam is directed through a second lens 108 and onto a mirror 110. The beam is reflected at the mirror 110 and reenters the beam splitter 106. A second half of the split initial beam is directed through a third lens 112 onto a tissue 114. Back-scattered light is collected for the lens 112 and enters the splitter 106. The combined light is then forwarded to a photodetector/analyzer 116, where an interference between the two beams is used to calculate a slope of the OCT signal. Thus, the system aims light at objects to be scanned using the sample beam existing the beam splitter. Light scattered from the tissue is combined with light returned from the reference arm, and a photodiode detects the resulting interferometric signal. Intereferometric signals can be formed only when the optical path length in the sample arm matches the reference arm length within coherence length of the source (10-15 μm). By gathering interference data at points across the surface, cross-sectional 2-D images can be formed in real time with resolution of about 10 μm at depths of up to one millimeter or deeper depending on the tissue optical properties [54-59].

Referring now to FIG. 1B, a fiber optics version of an OCT apparatus used in the examples set forth below generally, 150, is shown. Light from a superluminescent diode (SLD), a light source with low coherence, 152 can optionally passed through a first lens 154 into a first optical fiber or fiber bundle 155 and then directed to a 50/50 beam splitter 156. Half of the beam is directed into a second optical fiber or fiber bundle 157 and optionally through a second lens 158 and onto a mirror 160. The beam is reflected at the mirror 160 and into the optical fiber 157 and reenters the beam splitter 156. A second half of the split initial beam is directed into a third optical fiber or fiber 161 and optionally through a third lens 162 and then onto a tissue 164. Back-scattered light is then directed into the third optical fiber 161 or optionally through the third lens 162 and into then the third optical fiber 161 and then reenters the splitter 156. A portion of the backscattering beam and the reference beam are combined by the splitter 156 and forwarded through a fourth optical fiber 165 to a photodetector/analyzer 166, where an interference between the two beams is used to calculate a slope of the OCT signal. The apparatus 150 may also include an adaptive optics system 168 that modifies the sample beam to maximize or optimize the backscattered signal.

By averaging of the 2-D OCT images into a single 1-D composite OCT signal in depth, one can measure the optical properties of tissue or a specific tissue layer by analyzing the profile of the OCT signal. By varying the location of the 1-D composite OCT signal, a 3-D map of the tissue can be constructed with information about local profusion rate, local glucose concentration and local water concentration can be determined. The inventors have also found that certain structures within a tissue prove more reliable and reproducible OCT glucose concentration values. Thus, the method can also be used to determine those structures within a tissue or those tissues that can provide the most reliable and reproducible OCT glucose concentration values for continuous monitoring. In certain embodiments, the tissue is a mucosa, while in other embodiments the tissue structure is near a dermis-subdermis boundary and near a papillary and reticular junction in the dermis.

The inventors demonstrated that the higher resolution of OCT provides accurate and sensitive measurements of scattering from specific tissue layers. Moreover, due to coherent light detection, photons that are scattered from other tissue layers as well as diffusively scattered photons do not contribute to the OCT signal recorded from the tissue layer of interest. These features of the OCT technique provide accurate, sensitive, noninvasive, and continuous monitoring of blood glucose concentration with the proposed sensor.

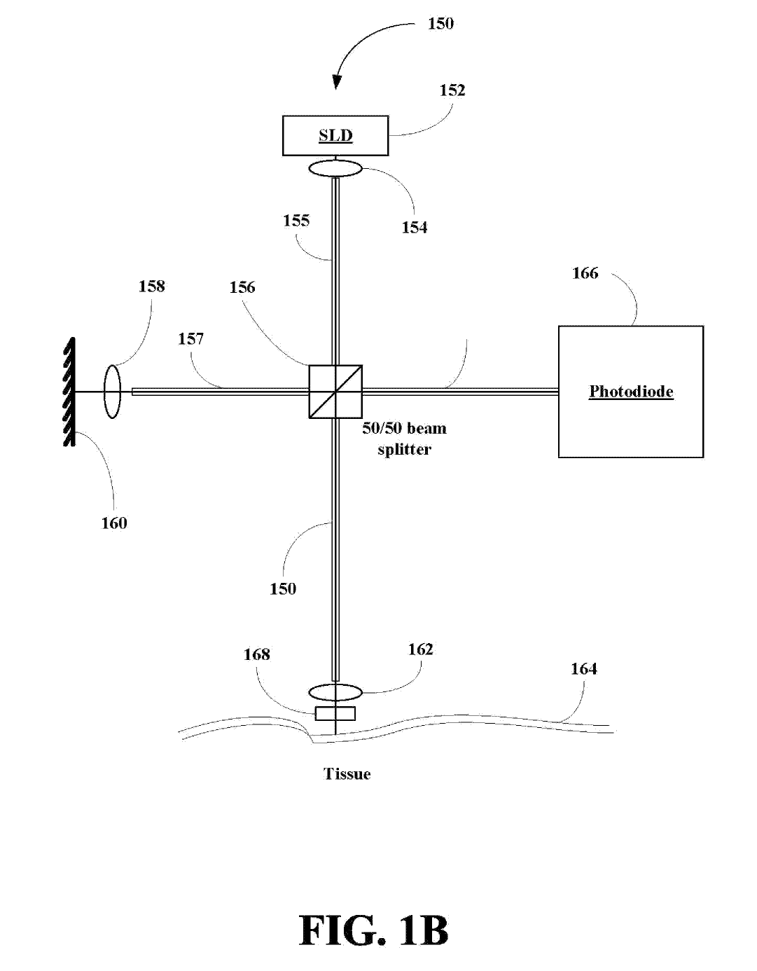

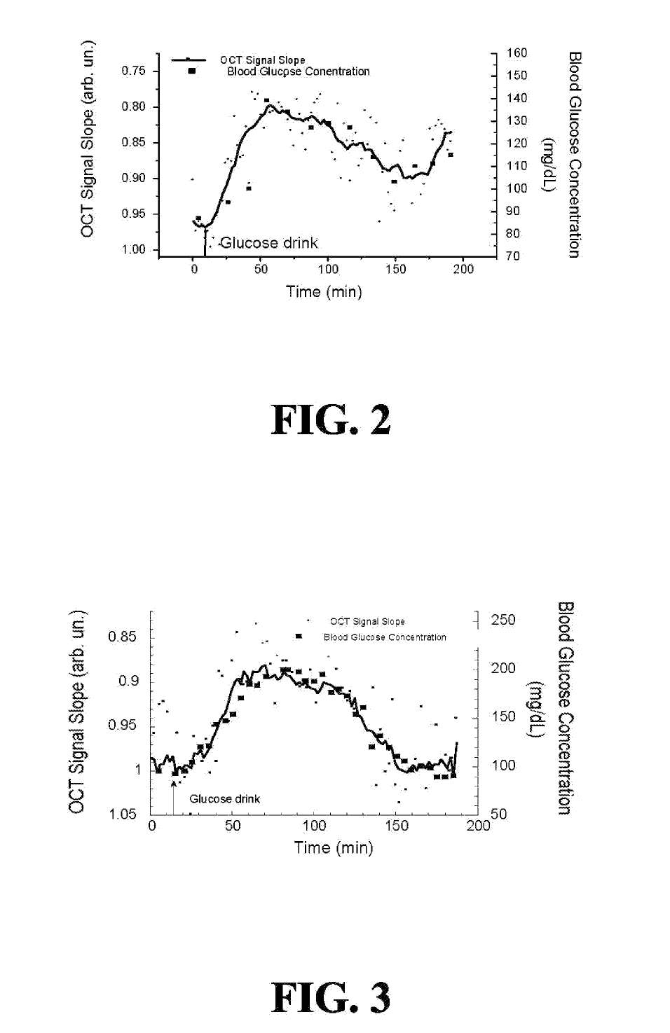

The inventors demonstrated in animal and clinical studies that the OCT technique is capable of continuous and noninvasive glucose monitoring when OCT signal slopes are measured from specific tissue layers [46-53]. Typical results obtained in clinical studies are shown in FIG. 2 and FIG. 3. The blood glucose concentration was measured each 15 and 5 minutes as shown in FIGS. 2 and 3, respectively, during the experiments. Decreases and increases of the OCT signal slope followed the changes in blood glucose concentration. The slopes were calculated at the depth of 550-600 μm as shown in FIG. 2 and 380-500 μm as shown in FIG. 3. The slopes changed significantly ˜17% with changes in glucose concentration from 90 to 140 mg/dL (first volunteer) and ˜15% with the changes in glucose concentration from 100 to 200 mg/dL (second volunteer).

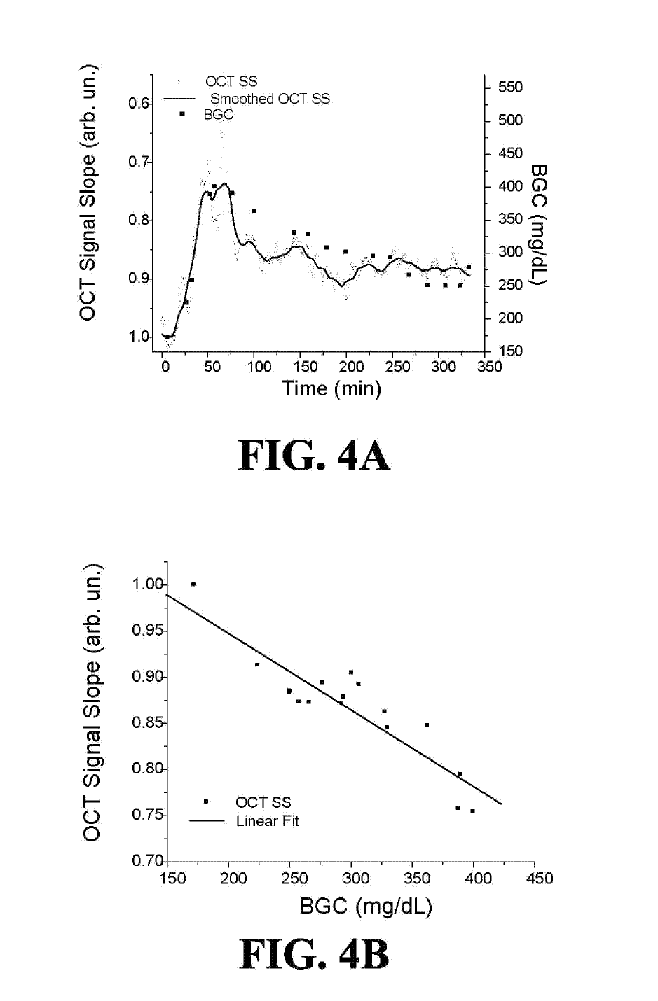

The inventors performed animal tests that included glucose clamping and square scanning of the beam over 0.2×0.2 mm (200μ×200μ) area of rabbit ear skin as shown in FIGS. 4A&B. Scanning over an area substantially reduced the scattering of the OCT data points compared to data obtained with linear scanning under similar conditions. Because areas have been shown experimentally improve OCT glucose measurement accuracy and precision by improving signal-to-noise ratio and other signal properties, a plurality of scans at specific locations within the area without scanning every location in the area will also give rise to improved OCT glucose measurements. Thus, the area scanning can be over the entire surface in any type of scanning pattern or the area scanning can be over patterned or randomally selected locations in the area.

The results of these studies demonstrated that 2-D lateral scanning of the incident OCT beam over an area such as a square provides better signal stability, reduces noise, and improves accuracy of the calculated glucose value. The 2-D lateral scanning can be performed over a rectangular, circular, elliptical, or any other 2-D area.

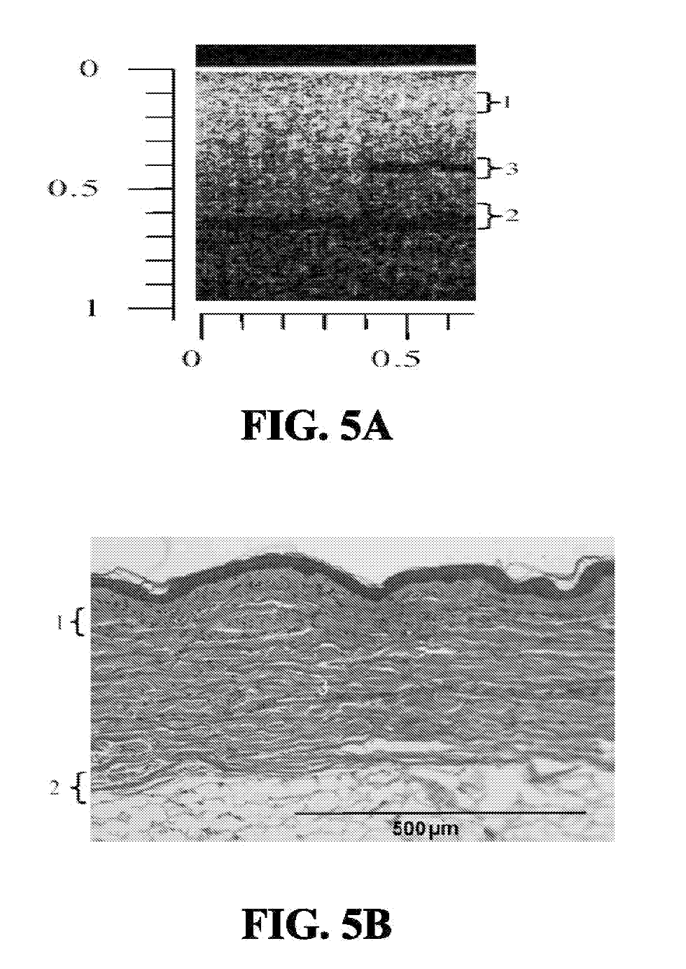

The inventors also identified specific skin layers in which an improved or best correlation between OCT signal slope and blood glucose concentration was obtained. The experiments were performed in young, 4-5 months old pigs (best model of human skin). Comparison between H&E-stained sections was performed to identify these layers on the OCT images as shown in FIGS. 5A&B that were used to map the OCT signals onto the H&E-stained section. Although the OCT signal slope correlated well with blood glucose concentration in all pigs, the best correlations between the OCT signal slope and blood glucose concentration occurred in specific depths within the tissue being imaged. A strong correlation between blood glucose concentration and OCT signal slope was found at the boundary between the dermis and subdermis. The OCT signal slope also correlated with blood glucose concentration in other skin layers, especially near the papillary and reticular junction in the dermis.

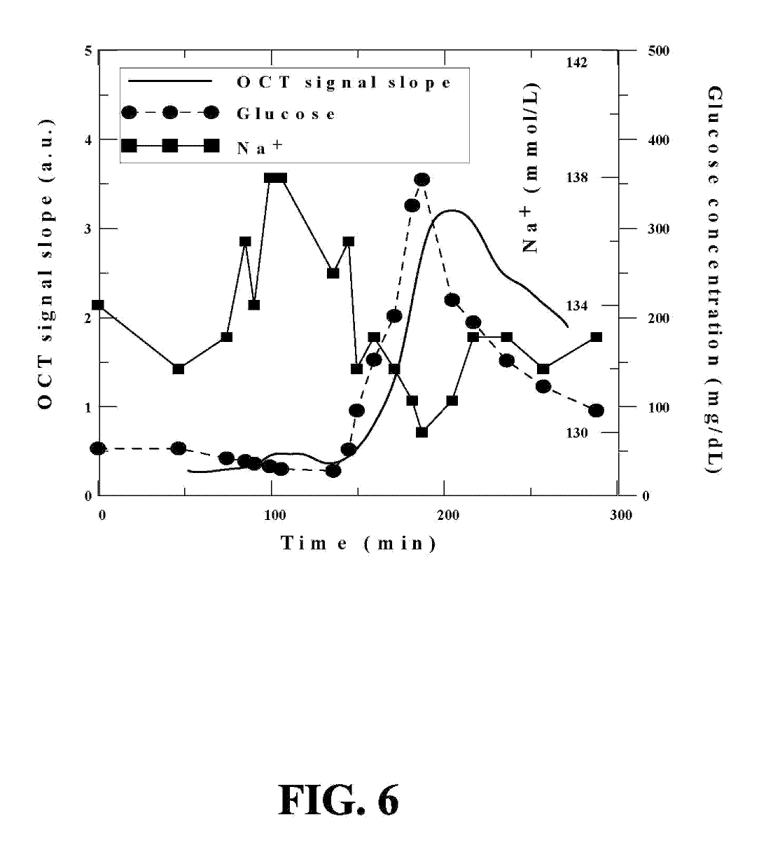

In another set of experiments, the inventors studied influence of other blood components of OCT signal slope, including major osmolytes such as Na⁺. In a pig study we injected NaCl and then glucose to compare their influence on OCT signal slope. This protocol simulates possible Na variation in diabetic and non-diabetic patients and, in particular, in critically ill patients with unstable concentration of Na⁺. The relative changes in OCT signal slopes induced by variation of blood glucose concentration were much greater than changes due to Na⁺ concentration variation. Averaged glucose-induced changes in OCT signal slope were five-fold greater than the changes in OCT signal slope associated with Na⁺ concentration variation in the physiological range as shown in FIG. 6.

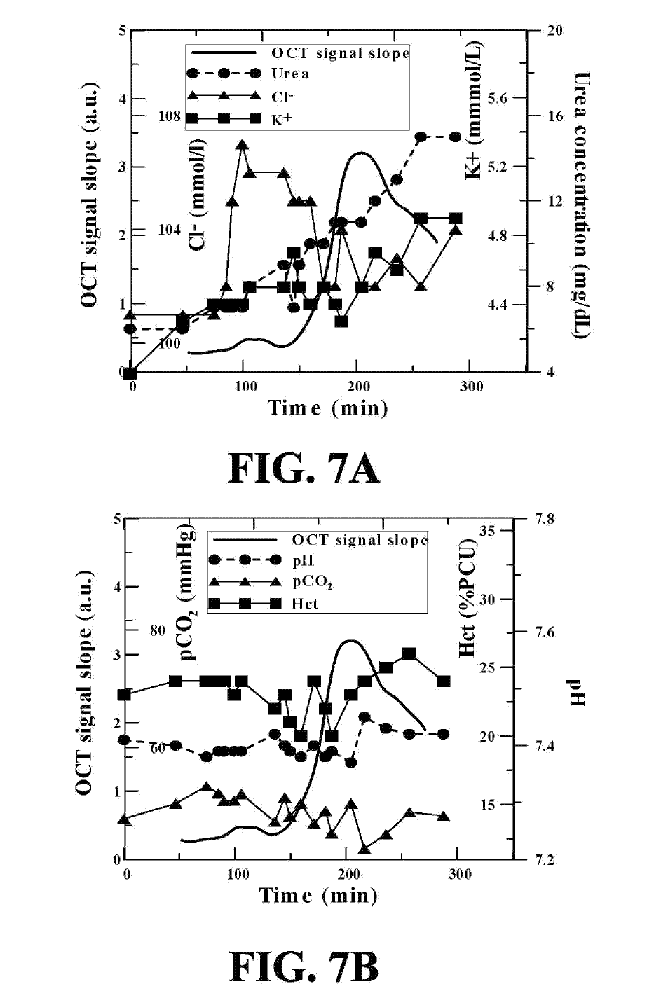

In general, analytes other than glucose and Na⁺ (i.e., Cl⁻, K⁺, Hct, pCO2, pH and urea) correlated poorly with the OCT signal slope as shown in FIGS. 7A&B. However, in one-half of the experiments, during infusion of sodium chloride, the OCT signal slope correlated with Cl⁻ (|R|=0.7-0.8). The data shown in FIG. 6 and FIGS. 7A&B demonstrate that the 2-D OCT signals correlate well with glucose concentration, but does not correlate with other analytes in the tissue.

In another study we have employed optical coherence tomography to quantitatively monitor hydration-induced changes in the corneal in-depth light backscatter distribution in real-time to visualize water movement within rabbit cornea in vivo following topical manipulating of corneal hydration using hypotonic or hypertonic agents.

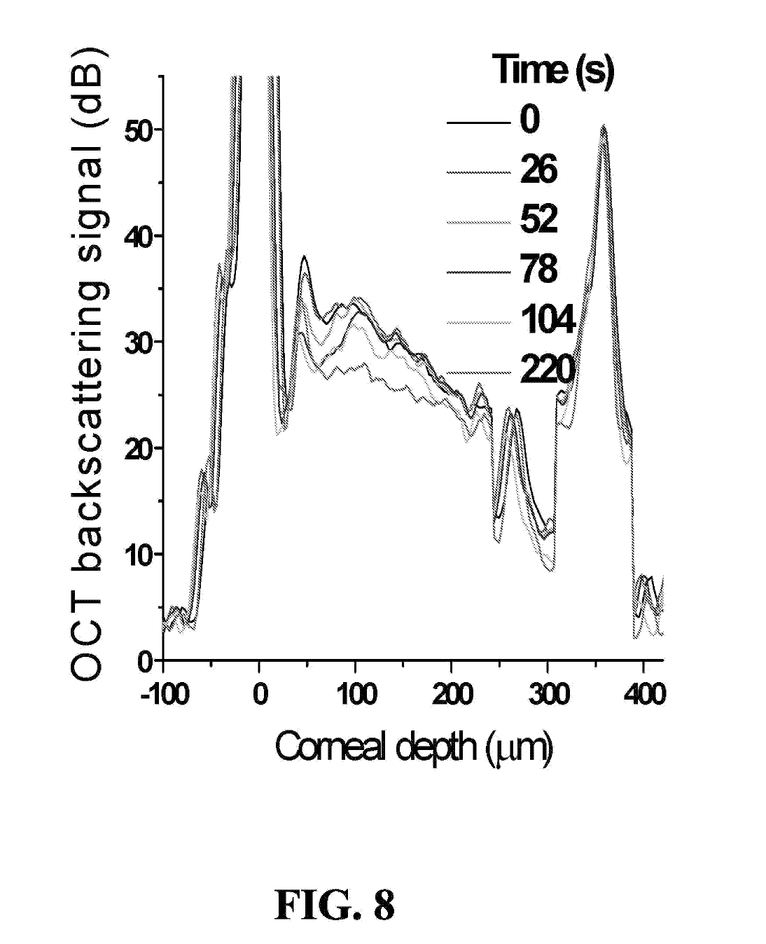

Referring now to FIG. 8, an experiment was conducted using 2-D OCT monitoring on a rabbit cornea in vivo to measure the transport of water within cornea following the application of one drop of water on the surface of rabbit cornea. In the figure, at time zero, the cornea was scanned under normal condition, immediately after this scan a drop of water was applied and additional scans were taken at subsequent time intervals. One can notice the change in OCT slope as well as the movement of the peak within cornea over time as cornea is becoming over hydrated.

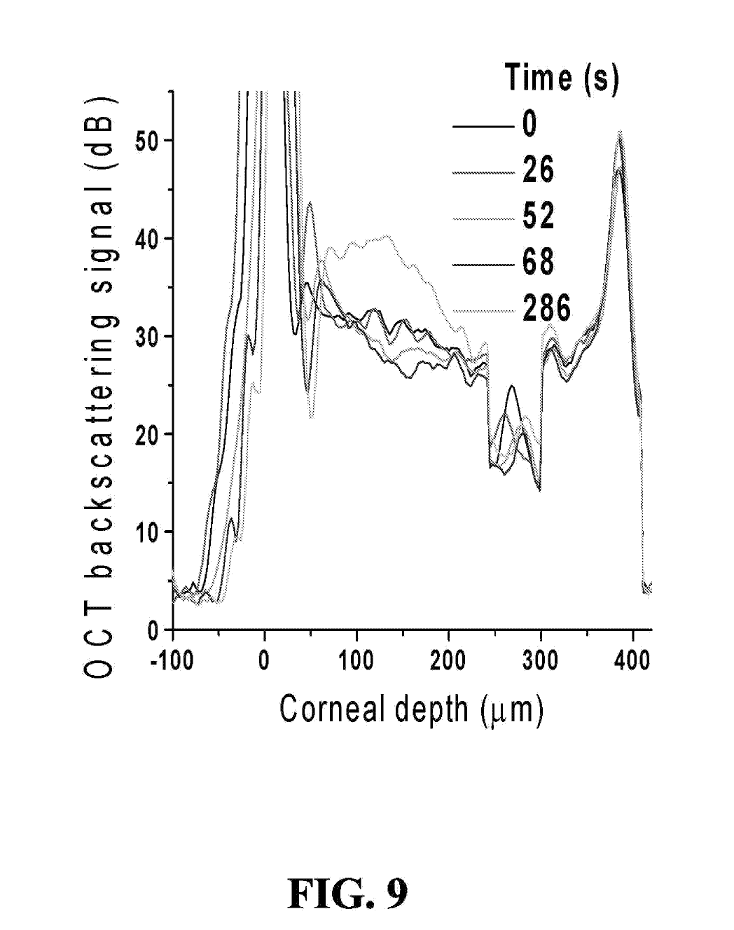

Referring now to FIG. 9, an experiment was conducted using 2-D OCT monitoring on a rabbit cornea in vivo to measure the transport of water within rabbit cornea in vivo following the application of one drop of dextrose which is known to cause cornea dehydration and shrinkage. In the figure, at time zero, the cornea was scanned under normal condition, immediately after this scan a drop of dextrose was applied and additional scans were taken at subsequent time intervals. One can notice the change in OCT slope as well as the movement of its peak within cornea over time as cornea is becoming dehydrated.

### REFERENCES CITED

The following literal references were cited in the above text. Most of the literal is merely background or evidences the novelty and non-obviousness of the subject matter of this application.
1. Wolfe R R, Allsop J R, Burke J F. Glucose metabolism in man: responses to intravenous glucose infusions. Metabolism 1979; 28: 210-20.
2. Wolfe R R, Herndon D N, Jahoor F, Miyoshi H, Wolfe M. Effect of severe burn injury on substrate cycling by glucose and fatty asids. N Engl J Med 1987; 317: 403-8.
3. Shangraw R E, Jahoor F, Miyoshi H, et al. Differentiation between septic and postburn insulin resistance. Metabolism 1989; 38: 983-9.
4. Mizock B A. Alterations in carbohydrate metabolism during stress: a review of the literature. Am J Med 1995; 98: 75-84.
5. McCowen K C, Malhotra A, Bistrian B R. Stress-induced hyperglycemia. Crit Care Clin 2001; 17: 107-24.
6. Fietsam R Jr, Bassett J, Glover J L. Complications of coronary artery surgery in diabetic patients. Am Surg 1991; 57: 551-7.
7. O'Neill P A, Davies I, Fullerton K J, Bennett D. Stress hormone and blood glucose response following acute stroke in the elderly. Stroke 1991; 22: 842-7.
8. Scott J F, Robinson G M, French J M, O'Connell J E, Alberti K G, Gray C S. Glucose potassium insulin infusions in the treatment of acute stroke patients with mild to moderate hyperglycemia: the Glucose Insulin in Stroke Trial (GIST). Stroke 1999; 30: 793-9.
9. Malmberg K, Norhammar A, Wedel H, Ryden L. Glycometabolic state at admission: important risk marker of mortality in conventionally treated patients with diabetes mellitus and acute myocardial infarction: long-term results from the Diabetes and Insulin-Glucose Infusion in Acute Myocardial Infarction (DIGAMI) study. Circulation 1999; 99: 2626-32.
10. Malmberg K. Prospective randomised study of intensive insulin treatment on long term survival after acute myocardial infarction in patients with diabetes mellitus. BMJ 1997; 314: 1512-15.
11. Malmberg K, Ryden L, Efendic S, et al. A randomized trial of insulin-glucose infusion followed by subcutaneous insulin treatment in diabetic patients with acute myocardial infarction (DIGAMI study): effects of mortality at 1 year. J Am Coll Cardiol 1995; 26: 57-65.
12. Takala J, Ruokonen E, Webster N R, et al. Increased mortality associated with growth hormone treatment in critically ill adults. N Engl J Med 1999; 341: 785-92.
13. Van der Berghe G, Wouters P, Weekers F, et al. Intensive insulin therapy in critically ill patients. N Engl J Med 2001; 345: 1359-67.
14. Bland J M, Altman D J. Statistical methods for assessing agreement between two methods of clinical measurement. Lancet, 8:307-10, 1986.
15. Capes S E, Hunt D, Malmberg K, Pathak P, Gerstein H C. Stress hyperglycemia and prognosis of stroke innondiabetic and diabetic patients: a systematic overview. Stroke 2001; 32: 2426-32.
16. Capes S E, Hunt D, Malmberg K, Gerstein H C. Stress hyperglycemia and increased risk of death after myocardial infarction in patients with and without diabetes: a systematic overview. Lancet 2000; 355:773-8.
17. Khaodhiar L, McCowen K, Bistrian B: Perioperative hyperglycemia, infection or risk? Curr Opin Clin Nutr Metab Care 1999; 2: 79-82.
18. Preiser J C, Devos P, Van Den B G. Tight control of glycemia in critically ill patients. Curr Opin Clin Nutr Metab Care 2002; 5: 533-7 R.
19. Van der Berghe G, Wouters P, Weekers F, et al. Reactivation of pituitary hormone release and metabolic improvement by infusion of growth hormone-releasing peptide and thyrotropin-releasing hormone in patients with protracted critical illness. J Clin Endocrinol Metab 1999; 84: 1311-23.
20. Van der Berghe G, Baxter R C, Weekers F, Wouters P, Bowers C Y, Veldhuis J D. A paradoxical gender dissociation within the growth hormone/insulin-like growth factor I axis during protracted critical illness. J Clin Endocrinol Metab 2000; 85: 183-92.
21. Schrier R W. Treating high-risk diabetic hypertensive patients with comorbid conditions. Am J Kidney Dis 2000; 36: S10-S17.
22. Hoogwerf B J. Tight blood glucose control: is it worth it? Cleve Clin J Med 1990; 57: 390-5.
23. Wang P H, Lau J, Chalmers T C. Meta-analysis of effects of intensive blood-glucose control on late complications of type I diabetes. Lancet 1993; 341: 1306-9.
24. Cunha-Vaz J. Lowering the risk of visual impairment and blindness. Diabet Med 1998; 15 Suppl 4: S47-S50.
25. Yki-Jarvinen H. Combination therapy with insulin and oral agents: optimizing glycemic control in patients with type 2 diabetes mellitus. Diabetes Metab Res Rev 2002; 18 Suppl 3: S77-S81.
26. Liebl A. Challenges in optimal metabolic control of diabetes. Diabetes Metab Res Rev 2002; 18 Suppl 3: S36-S41.
27. Oritz A, Ziyadeh F N, Neilson E G. Expression of apoptosis-regulatory genes in renal proximal tubular epithelial cells exposed to high ambient glucose and in diabetic kidneys. J Investig Med 1997; 45: 50-6.
28. Said G, Goulon-Goeau C, Slama G, Tchobroutsky G. Severe early-onset polyneuropathy in insulin-dependent diabetes mellitus: a clinical and pathological study. N Engl J Med 1992; 326: 1257-63.
29. Bernard G R, Vincent J-L, Laterre P-F, et al. Efficacy and safety of recombinant human activated protein C for severe sepsis. N Engl J Med 2001; 344: 699-709.
30. Rayfield E J, Ault M J, Keusch G T, Brothers M J, Nechemias C, Smith H. Infection and diabetes: the case for glucose control. Am J Med 1982; 72: 439-50.
31. Geerlings S E, Hoepelman A I. Immune dysfunction in patients with diabetes mellitus (DM). FEMS Immunol Med Microbiol 1999; 26: 259-65.
32. Rassias A J, Marrin C A, Arruda J, Whalen P K, Beach M, Yeager M P. Insulin infusion improves neutrophil function in diabetic cardiac surgery patients. Anesth Analg 1999; 88: 1011-6.
33. Losser M-R, Bernard C, Beaudeux J-L, Pison C, Payen D. Glucose modulates hemodynamic, metabolic, and inflammatory responses to lipopolysaccharide in rabbits. J Appl Physiol 1997; 83: 1566-74.
34. Jones R S, Putnam W, Andersen D K, Hanks J B, Lebovitz H E. Insulin's effect on bile flow and lipid excretion during euglycemia and hypoglycemia. Dig Dis Sci 1984; 29: 33-9.
35. Garcia-Marin J J, Villanueva G R, Esteller A. Diabetes-induced cholestasis in the rat: possible role of hyperglycemia and hypoinsulinemia. Hepatology 1988; 8: 332-40.
36. Cote G L, Fox M D, Northrop R B 1992 Noninvasive optical polarimetric glucose sensing using a true phase measurement technique IEEE Trans. Biomed. Eng. 39(7) 752-756.
37. King T W, Cote G L, McNichols R, Goetz Jr. M J 1994 Multispectral polarimetric glucose detection using a single Pockels cell Optical Engineering 33(8) 2746-2753.
38. Goetz Jr. M J, Cote G L, March W E, Erckens R, Motamedi M 1995 Application of a multivariate technique to Raman spectra for quantification of body chemicals IEEE Trans. Biomed. Eng. 42, 728-731.
39. Wicksted J P, Erkens R J, Motamedi M, March W E 1994 Monitoring of aqueous humor metabolites using Raman spectroscopy SPIE Proc. 2135 264-274.
40. Kohl M, Essenpreis M, Cope M 1995 The influence of glucose concentration upon the transport of light in tissue-simulating phantoms Phys. Med. Biol. 40 1267-1287.
41. Pan S, Chung H, Arnold M A, Small G W 1996 Near-infrared spectroscopic measurement of physiological glucose levels in variable matrices of protein and triglycerides Anal. Chem. 68 1124-1135.
42. Robinson M R, Eaton R P, Haaland D M, Koepp G W, Thomas E V, Stallard B R, Robinson P L 1992 Noninvasive glucose monitoring in diabetic patients: A preliminary evaluation Clin. Chem. 38 1618-1622.
43. Maier J S, Walker S A, Fantini S, Franceschini M A, Gratton E 1994 Possible correlation between blood glucose concentration and the reduced scattering coefficient of tissue in the near infrared Opt. Lett. 19(24) 2062-2064.
44. Gabriely I, Wozniak R, Mevorach M, Kaplan J, Aharon Y, Shamoon H 1999 Transcutaneous glucose measurement using near-infrared spectroscopy during hypoglycemia Diabetes Care 22(12) 2026-2032.
45. MacKenzie H A, Ashton H S, Spiers S, Shen Y, Freeborn S S, Hannigan J, Lindberg J, Rae P 1999 Advances in photoacoustic noninvasive glucose testing Clin. Chem. 45(9) 1587-1595.
46. Quan K M, Christison G B, MacKenzie H A, Hodgson P 1993 Glucose determination by a pulsed photoacoustic technique—an experimental study using a gelatin-based tissue phantom Phys. Med. Biol. 38(12) 1911-1922.
47. Esenaliev R O, Larin K V, Larina I V, Motamedi M 2001 Noninvasive monitoring of glucose concentration with optical coherence tomography Optics Letters, 26(13), 992-994.
48. Larin K V, Larina I V, Motamedi M, Gelikonov V, Kuranov R, Esenaliev R O 2001 Potential application of OCT for non-invasive monitoring of glucose concentration SPIE Proc. 4263 83-90.
49. Larin K. V., Esenaliev R. O., Petrova I. Y., Larina I. V., Eledrisi M. S., Motamedi M., Stuart C. A. Plasma Glucose Determination Using Optical Coherence Tomography: A Potential for Non-invasive Glucose Monitoring. The Endocrine Society's 83^{rd} Annual Meeting, (Denver, Col.—June 2001), pp. 225-226.
50. Larin K. V., Eledrisi M. S., Ashitkov T. V., Motamedi M., Esenaliev R. O. Monitoring of Tissue Optical Properties Using OCT: Application for Blood Glucose Analysis. SPIE Proc. 2002, v.4707, 181-188.
51. Larin K. V., Ashitkov T. V., Larina I. V., Petrova I. Y., Eledrisi M. S., Motamedi M., Esenaliev R. O. “Optical coherence tomography technique for noninvasive blood glucose monitoring: phantom, animal, and human studies”, Proc. SPIE, Vol. 4619, pp. 157-164, 2002.
52. Larin K. V., Eledrisi M. S., Motamedi M., Esenaliev R. O. Noninvasive Blood Glucose Monitoring with OCT Technique: A Pilot Clinical Study in Human Subjects. Diabetes Care, v.25(12), pp. 2263-2267, 2002.
53. Larin K V, Motamedi M, Ashitkov T V, Esenaliev R O Specificity of Noninvasive Blood Glucose Sensing Using Optical Coherence Tomography Technique: A Pilot Study, Physics in Medicine and Biology, v.48 pp. 1371-1390, 2003.
54. D. Huang, E. A. Swanson, C. P. Lin, J. S. Schuman, W. G. Stinson, W. Chang, M. R. Hee, T. Flotte, K. Gregory, C. A. Puliafito, and J. G. Fujimoto, “Optical coherence tomography,”Science 254, 1178-1181 (1991).
55. A. M. Sergeev, V. M. Gelikonov, G. V. Gelikonov, F. I. Feldchtein, K. I. Pravdenko, D. V. Shabanov, N. D. Gladkova, V. V. Pochinko, V. A. Zhegalov, G. I. Dmitriev, I. R. Vazina, G. A. Petrova, N. K. Nikulin, “In vivo optical coherence tomography of human skin microstructure,” Proc. SPIE 2328, 144-150 (1994).
56. S. N. Roper, M. D. Moores, G. V. Gelikonov, F. I. Feldchtein, N. M. Beach, M. A. King, V. M. Gelikonov, A. M. Sergeev, and D. H. Reitze, “In vivo detection of experimentally induced cortical dysgenesis in the adult rat using optical coherence tomography,” J. Neurosci. Meth. 80, 91-98 (1998).
57. G. J. Tearney, M. E. Brezinski, B. E. Bouma, S. A. Boppart, C. Pitris, J. F. Southern, and J. G. Fujimoto, “In vivo endoscopic optical biopsy with optical coherence tomography,” Science 276, 2037-2039 (1997).
58. A. M. Sergeev, V. M. Gelikonov, G. V. Gelikonov, F. I. Feldchtein, R. V. Kuranov, N. D. Gladkova, N. M. Shakhova, L. B. Snopova, A. V. Shakhov, I. A. Kuznetzova, A. N. Denisenko, V. V. Pochinko, Yu. P. Chumakov, and O. S. Strelzova, “In vivo endoscopic OCT imaging of precancer and cancer states of human mucosa,” Opt. Exp. 1, 432-439 (1997).
59. S. A. Boppart, G. J. Tearney, B. E. Bouma, J. F. Southern, M. E. Brezinski, and J. G. Fujimoto, “Noninvasive assessment of the developing Xenopus cardiovascular system using optical coherence tomography,” Proc. Natl. Acad. Sci. 94, 4256-4261 (1997).

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

We claim:

1. A method comprising the steps of:
generating radiation;
directing a first portion of radiation onto a plurality of locations of an area of a tissue site to generate back-scattered radiation corresponding to a plurality of 1-D OCT signals on a continuous or periodic basis,
directing a second portion of the radiation to a reflector to generate reference radiation on a continuous or periodic basis,
combining a portion of the back-scattered radiation and the reference radiation to form a combined radiation on a continuous or periodic basis,
forwarding the combined radiation to a detector to produce a plurality of optical coherence tomography signals on a continuous or periodic basis, and
calculating a glucose concentration using a composite slope of the optical coherence tomography signals on a continuous or periodic basis,
where the number of the plurality of signals is sufficient to improve the signal-to-noise ratio of a composite OCT signal improving the OCT derived glucose concentration.

2. The method of claim 1, wherein the area is of tissue is between about 200μ×200μ and about 2000μ×2000μ.

3. The method of claim 1, wherein a distance between pairs of locations in the area is between about 500 nm and 20 mm.

4. The method of claim 3, wherein the distance is between about 1 μm and about 10 mm.

5. The method of claim 1, wherein each scan is an in-depth scan.

6. The method of claim 1, wherein each scan is at a set penetration depth.

7. The method of claim 1, wherein each scan has a variable penetration depth.

8. The method of claim 1, wherein the area is regular or irregular.

9. The method of claim 1, wherein the plurality of locations comprises the entire area.

10. The method of claim 1, wherein the plurality of locations comprises a random selection of locations within the area.

11. The method of claim 1, wherein the plurality of locations comprises a patterned selection of locations within the area.

12. The method of claim 1, wherein the plurality of locations comprises a random selection of contiguous sub-areas within the area.

13. The method of claim 1, wherein the plurality of locations comprises a patterned selection of contiguous sub-areas within the area.

14. The method of claim 1, further comprising the step of:
detecting Doppler data, and
determining local blood profusion rates in the tissue.

15. The method of claim 5, further comprising the step of:
constructing 2-D images of each location.

16. The method of claim 15, further comprising the step of:
constructing a 3-D image of the area from the 2-D images at each location.

17. A method comprising the steps of:
generating first radiation having a first wavelength;
directing a first portion of first radiation onto a plurality of locations of an area of a tissue site to generate first back-scattered radiation corresponding to a plurality of 1-D OCT signals on a continuous or periodic basis,
directing a second portion of the first radiation to a reflector to generate first reference radiation on a continuous or periodic basis,
combining a portion of the first back-scattered radiation and the first reference radiation to form a first combined radiation on a continuous or periodic basis,
forwarding the first combined radiation to a detector to produce a plurality of first optical coherence tomography signals on a continuous or periodic basis,
generating second radiation having a second wavelength;
directing a second portion of second radiation onto a plurality of locations of an area of a tissue site to generate second back-scattered radiation corresponding to a plurality of 1-D OCT signals on a continuous or periodic basis,
directing a second portion of the second radiation to a reflector to generate second reference radiation on a continuous or periodic basis,
combining a portion of the second back-scattered radiation and the second reference radiation to form a second combined radiation on a continuous or periodic basis,
forwarding the second combined radiation to a detector to produce a plurality of second optical coherence tomography signals on a continuous or periodic basis, and
calculating a glucose concentration using data from a first composite OCT signal and a second OCT signal on a continuous or periodic basis,
where the number of the plurality of signals is sufficient to improve the signal-to-noise ratio of a composite OCT signal improving the OCT derived glucose concentration, where the first radiation is adapted to produce a high contrast OCT signal, where the second radiation is adapted to produce a water signal, and where data from the second radiation is used to reduce water artifacts during the calculating glucose concentration step.

18. The method of claim 17, where in the first wavelength is between about 700 nm and about 1300 nm and the second wavelength is between about 1300 nm and about 2000 nm.

19. A method comprising the steps of:
generating radiation having a first wavelength and a second wavelength;
directing a first portion of radiation onto a plurality of locations of an area of a tissue site to generate back-scattered radiation corresponding to a plurality of 1-D OCT signals on a continuous or periodic basis,
directing a second portion of the radiation to a reflector to generate first reference radiation on a continuous or periodic basis,
combining a portion of the back-scattered radiation and the reference radiation to form a first combined radiation on a continuous or periodic basis,
forwarding the combined radiation to a detector to produce a plurality of optical coherence tomography signals on a continuous or periodic basis,
calculating a glucose concentration using data from a first composite OCT signal and a second OCT signal on a continuous or periodic basis,
where the number of the plurality of signals is sufficient to improve the signal-to-noise ratio of a composite OCT signal improving the OCT derived glucose concentration, where the first radiation is adapted to produce a high contrast OCT signal, where the second radiation is adapted to produce a water signal, and where data from the second radiation is used to reduce water artifacts during the calculating glucose concentration step.

20. The method of claim 19, where in the first wavelength is between about 700 nm and about 1300 nm and the second wavelength is between about 1300 nm and about 2000 nm.

21. A method comprising the steps of:
2 generating radiation;
directing a first portion of radiation onto a plurality of locations of an area of a tissue site to generate back-scattered radiation corresponding to a plurality of 1-D OCT signals,
directing a second portion of the radiation to a reflector to generate first reference radiation,
combining a portion of the back-scattered radiation and the reference radiation to form a first combined radiation,
forwarding the combined radiation to a detector to produce a plurality of optical coherence tomography signals,
calculating a glucose concentration at each of a plurality of tissue depths using data from a first composite OCT signal, and
determining a tissue depth that generates a best OCT glucose concentration value,
where the number of the plurality of signals is sufficient to improve the signal-to-noise ratio of a composite OCT signal improving the OCT derived glucose concentration.

22. A method comprising the steps of:
generating radiation;
directing a first portion of radiation onto a plurality of locations of an area of a tissue site to generate back-scattered radiation corresponding to a plurality of 1-D OCT signals,
directing a second portion of the radiation to a reflector to generate first reference radiation,
combining a portion of the back-scattered radiation and the reference radiation to form a first combined radiation,
forwarding the combined radiation to a detector to produce a plurality of optical coherence tomography signals,
calculating analyte transport rates in the tissue or at the plurality of locations within the tissue area using data from a first composite OCT signal, and
determining a tissue depth that generates a best OCT glucose concentration value,
where the number of the plurality of signals is sufficient to improve the signal-to-noise ratio of a composite OCT signal improving the OCT derived glucose concentration.
